(19) United States
(12) Patent Application Publication
(10) Pub. No.: US 2005/0014735 A1
(43) Pub. Date: Jan. 20, 2005

(54) Treatment of Th2 dominated immunological disease states with progesterone receptor antagonists

(75) Inventors: Jeanette Alt, Burgwedel (DE); Bernd Eisele, Laatzen (DE)

Correspondence Address: CROWELL & MORING LLP;INTELLECTUAL PROPERTY GROUP, P.O. BOX 14300, WASHINGTON, DC, 20044-4300, US

(73) Assignee: Solvay Pharmaceuticals GmbH, Hannover (DE)

(21) Appl. No.: 10/855,790

(22) Filed: May 28, 2004

Related U.S. Application Data
(63) Continuation of application No. PCT/EP02/13290, filed on Nov. 26, 2002.
(60) Provisional application No. 60/334,122, filed on Nov. 30, 2001.

(51) Int. Cl. A61K 31/573

(52) U.S. Cl. 514/177

(57) ABSTRACT

A method of treating or inhibiting an immunological disease state which is associated with a Th2 dominated reaction pattern of the pathological immune response by administering to a patient in need thereof an effective amount of an antagonist for the human progesterone receptor.

### CROSS REFERENCE TO RELATED APPLICATIONS

This application is a continuation of international patent application no. PCT/EP02/13290, filed Nov. 26, 2002 designating the United States of America, and published in English as WO 03/045363 on Jun. 5, 2003, the entire disclosure of which is incorporated herein by reference. Priority is claimed based on U.S. provisional patent application No. 60/334,122, filed Nov. 30, 2001, the entire disclosure of which is also incorporated herein by reference.

### BACKGROUND OF THE INVENTION

The present invention relates to the novel use of progesterone receptor antagonists in the treatment and/or prophylaxis of immunological disease states which are associated with a Th2 dominated reaction pattern of the pathological immune response. In particular, the present invention relates to the novel use of non-endogenous progesterone receptor antagonists.

It is known that T helper cells (=Th cells) as essential components of the human cellular immunosystem are also playing a vital role in immunological disease states. Th cells can be differentiated in two different polarised forms which mobilise different types of effector responses, namely Th1 cells and Th2 cells. Th cells are able to produce and release (secret) cytokines. The information processing within the immune system largely depends on the production and release of cytokines.

With respect to their impact on the immune response, cytokines can be divided into two categories. Th1 cytokines (e.g. Tumor Necrosis Factor (=TNF) α, Interferone- (=IFN)-γ, Interleukin (=IL)-2, IL-12, IL-18), as secreted by Th1 cells, induce several cell-mediated cytotoxic and inflammatory reactions. A Th1 immune response pattern is therefore dominated by a cytotoxic and inflammatory profile of cytokine release. Th2 cytokines (e.g., IL-4, IL-5, IL-6, IL-10, IL-13), as secreted by Th2 cells, are associated with B cell antibody production. Th2 cytokines downregulate Th1-type reactivity. A Th2 dominated immune response pattern is therefore dominated by an anti-inflammatory profile of cytokine release.

Certain immunological disease states are known to be associated with a specific pattern of pathological immune response, typically referred to as “Th1 immune response pattern”. Among such immunological disease states are e.g. autoimmune diseases like rheumatoid arthritis and multiple sclerosis or states of acute rejection response after organ and/or bone marrow transplantation. Activated lymphocytes in the placenta and peripheral blood mononuclear cells (=PBMC) in the peripheral blood are known to react in an immunomodulatory, i.a. anti-abortive way. The production of non-inflammatory, non-cytotoxic interleukins (e.g., IL-3, IL-4, IL-10; “Th2 pattern”) is increased and the production of pro-inflammatory, cytotoxic cytokines (e.g., IFN-γ; TNFα; IL-2, “Th1 pattern”) is reduced. For example, in successful pregnancy the normal profile is a Th2-type immunity while a shift towards Th1-dominance is thought to be associated with unexplained habitual abortion.

It is known that endogenous progesterone inhibits in-vitro embryotoxic Th1 cytokine production totrophoblast in women with recurrent pregnancy loss (cf. B. C. Choi, K. Polgar, L. Xiao, J. A. Hill, Human Reproduction 15/1 (2000) 46-59). Progesterone does therefore seem to induce a shift from a Th1 immune response pattern to a Th2 immune response pattern in vitro. However, progesterone is not a highly selective endogenous messenger. For example, progesterone, besides binding to the human progesterone receptor, is known to compete with glucocorticoids at glucocorticoid receptors. Besides, current metabolites of progesterone (e.g. 12 [symbol] -hydroxyprogesterone, androstenedione, testosterone, desoxycorticosterone, 11-desoxycortisol, corticosterone, cortisol, 18-hydroxycorticosterone and aldosterone) have a number of quite different activities. In particular, the corticosteroidal metabolites have a profound effect on the immune system. These corticosteroids are a group of steroids that affect carbohydrate metabolism (gluconeogenesis, liver glycogen deposition, elevation of blood sugar), inhibit corticotropin secretion, and even possess pronounced anti-inflammatory activity. They also play a role in fat and protein metabolism, maintenance of arterial blood pressure, alteration of the connective tissue response to injury, reduction in the number of circulating lymphocytes, and functioning of the central nervous system.

### SUMMARY OF THE INVENTION

It has now surprisingly been found that dydrogesterone can influence Th1 dominated immune response patterns in a way that a stronger influence of a Th2 immune response pattern will prevail and that thereby dydrogesterone will exhibit a beneficial effect on immunological disease states which are characterised by a Th1 dominated immune response pattern.

Dydrogesterone (Duphaston®) is an orally active progestogen, which is similar to endogenous progesterone in its molecular structure but, unlike progesterone, acts as a highly selective progesterone receptor agonist. Besides, metabolites of dydrogesterone are known to be either also selective agonists of human progesterone receptors or to be inactive. It can therefore be deduced that any physiological effects triggered by dydrogesterone will normally be mediated via the progesterone receptor. In turn, blocking of the human progesterone receptor will lead to the opposite effect and will influence Th2 dominated immune response patterns in a way that a stronger influence of a Th1 immune response pattern will prevail and that thereby antagonists of the human progesterone receptor will exhibit a beneficial effect on immunological disease states which are characterised by a Th2 dominated immune response pattern.

The invention therefore relates to the use of progesterone receptor antagonists, in particular the use of non-endogenous progesterone receptor antagonists, for the treatment and/or prohylaxis of an immunological disease state which is associated with a Th2 dominated reaction pattern of the pathological immune response.

Suitable non-endogenous progesterone receptor antagonists include those selected from the group consisting of mifepristone (RU486); onapristone; antiprogestin J956; 5H-progesterone metabolites and their analogues 16 [symbol] ,17 [symbol] -cyclohexan-5H-pregnan-3,20-diones; antiprogestogen Org 31710, antiprogestogen Org 33628; antiprogestin ZK 137316; antiprogestin ZK 230211; antiprogestin ZK 98299; dexamethasone-mesylate (Dex-Mes); dexamethasone-oxetanone (Dex-Ox); ZM 172406 and the R enantiomer of ZM 150271. Further compounds which may be suitable as non-endogenous progesterone receptor antagonists according to the invention are the so-called mesoprogestins. Mesoprogestins (also called progesterone receptor modulators), like mesoprogestin J1042, take an intermediate position between progestogens and antiprogestogens. The suitability of mesoprogestins as non-endogenous progesterone receptor antagonists has to be assessed on a case by case basis.

Mifepristone is known to have immune modulating properties. Mifepristone is also known to be a glucocorticoid antagonist and to bind to the glucocorticoid receptor with an affinity fourfold greater than that of dexamethasone. Mifepristone has an affinity to androgen receptors as well and possesses weak antiandrogenic activity. It is well known that the androgen receptor mediates effects on the immune system. Androgens suppress both T-cell and B-cell immune responses. Mere observations of immune modulating properties of mifepristone are therefore not indicative for the progesterone receptor mediated effects of mifepristone according to the invention.

Immunological disease states which are associated with a Th2 dominated reaction pattern of the pathological immune response and which can be treated by non-endogenous progesterone receptor antagonists according to the invention comprise autoimmune diseases, especially systemic lupus erythematosus. Systemic sclerodermia and sjogren syndrome are disease states that are currently cited as being dominated by a Th2 immune response pattern but scientific opinions also exist that they may be disease states that are dominated by a Th1 immune response pattern. Immune myocarditis (coxsackie virus Type B-3) on the other hand is a disease state that is currently cited as being dominated by a Th1 immune response pattern but scientific opinions also exist that it may be a disease state that is dominated by a Th2 immune response pattern.

Further immunological disease states which are associated with a Th2 dominated reaction pattern of the pathological immune response and which can be treated by non-endogenous progesterone receptor antagonists according to the invention comprise chronic rejection reactions after transplantation, especially chronic rejection reaction against transplanted organs and/or tissue and against transplanted bone marrow (Host versus Graft), chronic Graft versus Host rejection reaction and chronic rejection reaction in xenotransplantation and other diseases like allergy, asthma bronchiale and chronic allergic rhinitis.

The non-endogenous progesterone receptor antagonists according to the invention are capable of binding to the human progesterone receptor and by blocking said receptor can induce a shift from a Th2 dominated immune response pattern to a Th1 dominated immune response pattern. Further evidence for this proposed mechanism and the presently unacknowledged role of antagonists/agonists of the human progesterone receptor (like, also, endogenous progesterone) in immunology is given by the following observations: autoimmune diseases vary in the course of a menstrual cycle when progesterone levels vary. Symptoms of multiple sclerosis and rheumatoid arthritis worsen shortly before start of menstrual bleeding. This happens in parallel to the involution of corpus luteum when the progesterone levels are declining. Autoimmune diseases vary during and also after pregnancy. Rheumatoid arthritis often improves in the course of pregnancy and worsens again after delivery. The same holds true for multiple sclerosis. In systemic lupus erythemathosus and systemic sclerodermia the opposite is the case. With these diseases, symptoms are often worsening during pregnancy.

Progesterone receptor agonists and/or antagonists also play an important role during pregnancy. The fetal-placental unit is a semi-allograft because of the paternal genetic contribution. Subsequently, there is a maternal immune response to the allogeneic pregnancy. The constituents of the maternal immune reaction to the allogeneic stimulus are not different from any other immune reaction and allogeneic conceptus (trophoblast) is in principle like all other allogeneic tissue grafts. The immunologic recognition of pregnancy and the subsequent activation of the maternal immune system is necessary for a successful pregnancy. It results in an upregulation of progesterone receptors on activated lymphocytes among placental cells and decidual CD56+ cells. In unexplained cases of pregnancy loss, the most practical and promising way of therapeutic intervention is the direct interaction with the progesterone receptor by dydrogesterone (Duphaston®).

One reason why progesterone receptor mediated pathological immune responses are not confined to women but can also occur in men is chimerism, more exactly microchimerism. Chimerism indicates that a body contains cell populations derived from different individuals and microchimerism denotes low levels of chimerism (e.g. blood cells, fetal cells). Pregnancy induces microchimerism. Fetal cells can be detected in the circulation of the mother as early as five weeks of gestation. As fetal cells contain paternal material (i.e., foreign material that is derived from the father) they are able to induce an immune reaction. Pathological processes induced by and associated with the subsequent immune reactions will then lead to the sequelae of autoimmune disease. The link to pregnancy is the main reason for the increased prevalence of autoimmune diseases in women. But also in males the reasons for the development of autoimmune diseases can be explained by the processes of microchimerism. Here, however, the inducing factors are different. In males (but also in nullipara females) microchimerism can be induced via different routes, like blood transfusion (whole blood or packed red cells, PRCs), transplantation (in organ recipient), resorption (engraftment) of a twin in utero or via mother derived cells that get access into the blood circulation of the patient already in utero or during labour. These cells are known to persist over years in the patient. Microchimerism cells are not only found in the blood circulation but also in organs of patients (e.g., in the skin of patients suffering from systemic sclerodermia). Further, transplanted organs reveal progesterone receptors on their surface after transplantation. Progesterone favours a shift towards a Th2 immune response and thus, antagonizes the development and prevalence of a Th1 response. Pregnancy is characterized by a Th2 response. The Th2 response is initiated by the interaction of the fetal cells of the trophoblast with the mother. During this interaction lymphocytes and CD56+ decidual cells in the uterus but also peripheral blood mononuclear cells are upregulating progesterone receptors on their surface. In diseases which are induced by microchimerism, this basic mechanism is also followed, and finally causes the pathological immune response.

According to the invention, the antagonists for the human progesterone receptor are therefore suitable for the treatment and/or prophylaxis of immunological disease states which are associated with a Th2 dominated reaction pattern of the pathological immune response in mammals, preferably humans, of either gender.

The effects according to the invention of non-endogenous antagonists for the human progesterone receptor on Th2 dominated immune response patterns can be shown indirectly by showing the effects of dydrogesterone on Th1 dominated immune response patterns in pharmacological tests as set forth below.

Pharmacological Test Method

The effects of dydrogesterone on Th1 and Th2 cytokine profiles produced by mitogen-stimulated peripheral blood mononuclear cells (=PBMC) were determined.

Several studies have pointed to a deviation of cytokine profiles in women when during pregnancy. Pregnancy is associated with a shift towards Th2 bias, evinced by changes in peripheral T cells, by cytokine production by peripheral lymphocytes and by cytokine changes at the maternal-fetal interface. Unexplained recurrent spontaneous abortion (URSA) seems to be associated with a stronger bias towards Th1 cytokines. Women who have earlier been gone through URSA may therefore be regarded as a pre-selected group of patients that will typically respond to an immunological stimulation by showing a Th1 dominated pattern of immune response. The pharmacological test was therefore performed with a suitable URSA group which comprised of women admitted with spontaneous abortion for evacuation, (i) who have had at least two previous unexplained miscarriages, (ii) who were currently undergoing at least a 3rd abortion and (iii) who had been fully investigated.

The relationship between habitual abortion and cytokine production response in 54 women with normal pregnancy (and at least 3 previous normal pregnancies) and 23 women with a history of habitual abortion of unexplained aetiology (URSA group) has been investigated.

Mitogen-induced stimulation of Peripheral Blood Mononuclear Cells (PBMC):

Peripheral blood was obtained by venipuncture from the abortion group on the day of abortion. PBMC is separated by Ficoll-paque (Pharmacia Biotech, Sweden) density gradient centrifugation, suspended in RPMI medium (=“Roswell Park Memorial Institute”-medium; obtained from GIBCO/BRL, USA) containing 10% fetal calf serum, aliquoted into 96 well tissue culture plates at a density of 100.000 cells per well and then stimulated with the mitogen phytohemagglutinin (=PHA, Sigma Chemicals, USA) at a concentration of 5 mg/ml for a period of 96 hours, with autologous placental antigens or with trophoblast antigens. Progesterone, dydrogesterone (Duphaston®) and RU486 (=mifepristone) was added in different combinations as described below. Progesterone was tested at concentrations of 10⁻³ ml/L, 10⁻⁵ ml/L and 10⁻⁷ ml/L. RU486 was tested at equivalent concentrations. Culture supernatants were harvested at 96 hours for determination of the levels of cytokines secreted by the PBMC into the supernatants. Cytokine levels were determined in the supernatants by enzyme-linked immunosorbent assay (=ELISA).

Protocol of Experimental Samples:

Each sample was tested in the following manner:
PBMC+mitogen
PBMC+mitogen+progesterone
PBMC+mitogen+progesterone+RU486
PBMC+mitogen+dydrogesterone
PBMC+mitogen+dydrogesterone+RU486
PBMC+mitogen+Carrier
PBMC+mitogen+Carrier+RU486

Determination of cytokine levels by ELISA:

Th1 cytokines TNF-α and IFN-γ, and Th2 cytokines IL-4, IL-6 and IL-10 were evaluated in these samples by ELISA using kits that were obtained from Immunotech SA, France. These consisted of “sandwich ELISA”; briefly the first step leads to the capture of the relevant cytokine by monoclonal anti-cytokine antibodies bound to the wells of microtiter plates. In the second step, a second biotinylated monoclonal is added together with streptavidin-enzyme (peroxidase or alkaline phosphatase) conjugate. The biotinylated antibody binds to the solid phase antibody-antigen complex, and in turn, binds the conjugate. After incubation the wells are washed and the binding of the streptavidin-enzyme via biotin is followed by the addition of a chromogenic substrate. The intensity of the coloration produced is proportional to the concentration of the cytokine in the sample. Standard curves are plotted for each of the cytokines using reference recombinant cytokines and the results are read from these curves. The sensitivity of each of the assays is as follows: 5 pg/ml of TNF-α, 0.08 IU/ml of IFN-γ, 5 pg/ml of IL-4 and 5 pg/ml of IL-10. Results will be analysed by the t-test for independent samples.

There were significant differences between normal pregnant women and women with habitual abortion in terms of the levels of cytokines. Women with a normal pregnancy showed a higher Th2 bias, whilst women with a history of habitual abortion showed a bias towards Th1 reactivity. Results of the ex-vivo experiments according to the pharmacological test report given above showed that the administration of dydrogesterone (Duphaston®) in women with spontaneous abortion and habitual abortion reduced the activity of lymphocytes, resulted in a shift from a Th1 towards a Th2 bias (as evidenced by the reduction of IFN-γ and a marked increase in Interleukins 4 and 6) and in the increased release of progesterone induced blocking factor (PIBF), a 34 kDA protein. PIBF is a molecular mediator within the subsequent immune response.

The results of the pharmacological test method give above can also be confirmed in an in vivo test model involving pregnant mice.

The non-endogenous progesterone receptor antagonists according to the invention may be administered in conventional pharmaceutical preparations. The doses to be used may vary individually and will naturally vary according to the type of condition to be treated and the substance used.

The progesterone receptor antagonists may be contained together with conventional pharmaceutical auxiliaries and/or carriers, in solid or liquid pharmaceutical preparations. Examples of solid preparations are preparations which can be administered orally, such as tablets, coated tablets, capsules, powders or granules, or alternatively suppositories. These preparations may contain conventional pharmaceutical inorganic and/or organic carriers, such as talcum, lactose or starch, in addition to conventional pharmaceutical auxiliaries, for example lubricants or tablet disintegrating agents. Liquid preparations such as suspensions or emulsions of the active substances may contain the usual diluents such as water, oils and/or suspension agents such as polyethylene glycols and the like. Other auxiliaries may additionally be added, such as preservatives, taste correctives and the like.

The active substances may be mixed and formulated with the pharmaceutical auxiliaries and/or carriers in known manner. For the production of solid medicament forms, the active substances may for example be mixed with the auxiliaries and/or carriers in conventional manner and may be wet or dry granulated. The granules or powder can be poured directly into capsules or be pressed into tablet cores in conventional manner. These can be coated in known manner if desired.

Further examples of suitable pharmaceutical preparations include preparations for topical and/or transdermal delivery like gels, ointments or transdermal patches, devices and/or preparations for intravaginal administration, formulations for intranasal administration like sprays or formulations suitable for injection like depot injections or implants.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A method of treating or inhibiting an immunological disease state which is associated with a Th2 dominated reaction pattern of the pathological immune response, said method comprising administering to a patient In need thereof an effective amount of a progesterone receptor antagonist.

2. A method according to claim 1, wherein the progesterone receptor antagonist is a non-endogenous progesterone receptor antagonist.

3. A method according to claim 1, wherein the disease state is an autoimmune disease.

4. A method according to claim 3, wherein the autoimmune disease is systemic lupus erythematosus.

5. A method according to claim 3, wherein the autoimmune disease is systemic sclerodermia or Sjogren's syndrome.

6. A method according to claim 3, wherein the autoimmune disease is immune myocarditis (coxsackie virus Type B-3).

7. A method according to claim 1, wherein the disease state is a chronic rejection reaction after transplantation.

8. A method according to claim 7, wherein the disease state is selected from the group consisting of chronic rejection reaction against transplanted organs, tissue or bone marrow (Host versus Graft); chronic Graft versus Host rejection reaction, and chronic rejection reaction in xenotransplantation.

9. A method according to claim 1, wherein the disease state is selected from the group consisting of allergy, asthma bronchiale and chronic allergic rhinitis.

10. A method according to claim 1, wherein the disease state is selected from the group consisting of typhus (rickettsia prowazeki), legionaire's disease (legionella pneumophila), lepromatous leprosy, Epstein Barr virus infection, infection with parasites and parasitic worms, and leishmaniosis (leishmania major).

11. A method according to claim 10, wherein the disease state is infection with schistosomia mansoni.

12. A method according to claim 2, wherein the non-endogenous progesterone receptor antagonist is selected from the group consisting of mifepristone (RU486); onapristone; antiprogestin J956; 5H-progesterone metabolites and analogs thereof; 16 [symbol] ,17 [symbol] -cyclohexan-5H-pregnan-3,20-diones; antiprogestogen Org 31710, antiprogestogen Org 33628; antiprogestin ZK 137316; antiprogestin ZK 230211; antiprogestin ZK 98299; dexamethasone-mesylate (Dex-Mes); dexamethasone-oxetanone (Dex-Ox); ZM 172406, and the R enantiomer of ZM 150271.

13. A method according to claim 1, wherein the progesterone receptor antagonist is a mesoprogestin.

14. A method of ameliorating an immunological disease state which is associated with a Th2 dominated reaction pattern of the pathological immune response in a mammal, sad method comprising administering to a mammal in need thereof an effective amount of a progesterone receptor antagonist.

15. A method according to claim 14, wherein said progesterone receptor antagonist is a non-endogenous progesterone receptor antagonist.

16. The method of claim 14, wherein said mammal is female.

17. The method of claim 14, wherein said mammal is male.
